(19) United States
(12) Patent Application Publication
(10) Pub. No.: US 2020/0077949 A1
(43) Pub. Date: Mar. 12, 2020

(54) SYSTEM AND METHOD FOR THE MONITORING OF THE METABOLIC ENERGY SYSTEMS AND THE STATUS OF THE AUTONOMIC NERVOUS SYSTEM

(71) Applicant: ARGUSCAN KFT., Budapest (HU)

(72) Inventor: Péter BEZZEG, Budapest (HU)

(21) Appl. No.: 16/311,758

(22) PCT Filed: Jul. 3, 2017
(86) PCT No.: PCT/IB2017/054011

§ 371 Date: Mar. 29, 2019

(30) Foreign Application Priority Data
Jul. 4, 2016 (HU) P1600404

(51) Int. Cl. A61B 5/00; A61B 5/0205; A61B 5/11

(52) U.S. Cl. CPC A61B 5/4866; A61B 5/02055; A61B 5/4035; A61B 5/6823; A61B 2503/10; A61B 2503/40; A61B 5/1112; A61B 5/1118

(57) ABSTRACT

The subject of the invention is a sports physiological measuring system and method for the monitoring of the aerobic and anaerobic metabolic energy generating system and the autonomic nervous system, primarily during physical exercise.

The subject of the invention is a sports physiological measuring system and method for the monitoring of the aerobic and anaerobic metabolic energy generating system and the autonomic nervous system, primarily during physical exercise.

### BACKGROUND OF THE INVENTION

During physical exercise, the operation of the aerobic and anaerobic metabolic system under the control of the autonomic nervous system provides the energy required for motion. FIG. 1 illustrates the operation of the energy generating system during sporting activities.

During endurance and power-endurance trainings and competition, mainly the aerobic and anaerobic lactic acidic energy system provides the necessary energy. The operating ratio of the two energy-generating systems depends on the current performance and intensity. While continuously performing training, the aerobic system dominates at lower intensities and the anaerobic energy production increases at higher intensities. The aerobic nature of energy production is shown by the parallel change in oxygen (O₂) consumption and carbon dioxide (CO₂) emission of the athlete (either increasing or decreasing). However, when intensity increases and the carbon dioxide emission is not proportional to the increasing oxygen consumption but the carbon dioxide emission is higher, this indicates that the energy production has switched to the anaerobic character. When the energy production switches from aerobic to anaerobic, this obtained intensity value is the Anaerobic Threshold (AT).

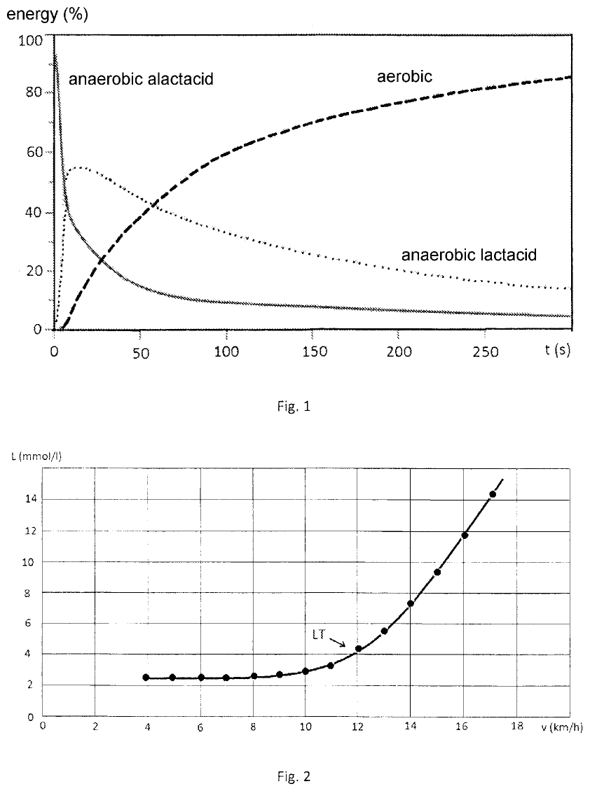

Another important physiological parameter connected to sporting activities is the Lactate Threshold (LT). The lactate content of blood changes during sporting depending on the intensity. The lactate content increases with increasing intensity and decreases with decreasing intensity. FIG. 2 shows the lactate curve of a runner, the horizontal axis representing the speed of the runner as intensity and the vertical axis showing the blood lactate concentration. The intensity above which the lactate concentration suddenly increases is the LT point. Generally, this is around 4 mmol/l and frequently equals to the AT intensity value. The AT parameter can be measured in the laboratory using the ergospirometric test. The lactate content of blood can be measured using a hand held lactate meter during any kind of training. In everyday practice the trainers consider the well measurable 4 mmol/l LT level as AT.

Untrained or badly trained athlete becomes unstable at intensities higher than AT, the lactate level in blood rapidly increases leading to a high degree feeling of fatigue, muscle stiffness thus leading to the decrease of the intensity. However, a well-trained athlete is capable of working above the AT level for a longer period of time, i.e. the athlete can accomplish the racing or training distance at an intensity above AT. It is the preparedness and training conditions that determine the maximum intensity at which one can accomplish the racing or training distance. The athlete prepares for his/her racing distance with a previously determined racing speed. At the beginning of the distance all athletes are capable of the target racing speed, however, at the end only those are capable who possess proper endurance. Endurance is proportional to the aerobic capacity and speed is proportional to the anaerobic capacity. The goal is that the athlete could accomplish the racing distance with the necessary anaerobic capacity, i.e. speed, in turn with the required aerobic capacity, i.e. endurance.

When the coach prepares the athlete for a definite race distance, he/she develops the aerobic and anaerobic capacities with different special exercises. When the athlete carries out the trainings at or near to the racing speed, the ratio between the aerobic and anaerobic energy generation systems has not been measurable and this problem, in fact, has not been addressed so far. It depends on the preparedness of the athlete in what aerobic/anaerobic energy generation ratio one can accomplish the racing distance. From the outside, there are no signs of the aerobic/anaerobic ratio, i.e. the stop watch does not show any difference when the time provided to cover the distance is identical, however, it is this ratio that determines the efficacy of training, feeling of fatigue and regenerative capabilities. From training to training, forcing the inadequate aerobic/anaerobic ratio may lead to the decrease of capabilities and possible over training.

In the course of trainings the continuous monitoring of the aerobic/anaerobic ratio has not been solved, the preparation has not been objective enough, it has been based on habits and intuitions, frequently trusting in luck when preparing competitors. The invention provides opportunity to the continuous monitoring of the activities of the aerobic/anaerobic energy producing systems that is an essential condition to accurately prepare the athlete for races.

The aerobic and anaerobic systems produce the energy required for athletic performance but depending on the distance and intensity of the training or the race the ratio of their activities may vary. Any training that increases or decreases the operation of one of the systems will influence that of the other one. The goal is to maximize the aerobic system to an optimal anaerobic system. During the preparation of the athlete the ideal ratio of the two systems has to be determined for the certain race distance.

In the course of daily trainings it has represented a great problem not having the opportunity to continuous monitor the ratio of the two energy-producing systems in the various intensity sections of trainings. This is important, because the increased improvement of the aerobic or anaerobic systems may weaken the other, thus, the targeted ratio may be shifted during the preparation and the performance of the racing distance may not be the most efficient, not of the highest speed.

In endurance and power-endurance sports regarding the operation of the aerobic and anaerobic systems, ergospirometry, as well as lactate concentration of blood during training determination are usually carried out. For ergospirometric tests, the VO₂ max. (giving the maximum quantity of oxygen that can be taken up and transported, also called maximum oxygen uptake), and the anaerobic threshold (AT) are the two most important parameters but with the exception of cycling and running, it is hard or impossible to carry out the tests in the environment of the original motion (swimming, kayak-canoe, etc.), therefore, the use of the results is limited. The VO₂ max is the maximum oxygen uptake capacity showing the capacity of the aerobic system, the anaerobic threshold (AT) is the performance limit above what the anaerobic system dominates. During training, there is the possibility to check the anaerobic threshold by lactate measurement that is an important parameter of trainedness, however, this is a slowly changing parameter taking weeks, thus it indicates the incorrectness of the training protocol or the shifting of the ratio of the aerobic/anaerobic energy production system of the athlete into the wrong direction only with a significant delay. Thus, the lactate measurement does not provide an appropriate feedback for every day's work, the slow process of changing capability.

The autonomic nervous system controls the operation of the metabolic energy production system. The change of the sympathetic and parasympathetic activity greatly influences the operation of the energy producing system. For stronger sympathetic tones, the aerobic system starts up with more difficulty, warm-up is slower, the aerobic system is more sensitive to anaerobic overloads, the energy producing system responds to speed changes quickly and with intensive anaerobic reflexes, it is able to enter higher pulse ranges but regeneration between sections of the race distance is slower. The strengthening of the parasympathetic tone promotes the fast warm-up, the aerobic system is capable of operating at higher levels that helps the long lasting, continuous work but to the contrary, badly and slowly responds to speed changes, pulse increases slowly and maximum achievable pulse decreases together with the achievable maximum speed.

The fact that an athlete responds better to the aerobic or the anaerobic practices greatly depends on the inherited characteristics of the autonomic nervous system that is understood as the ratio of sympathetic to parasympathetic behaviour. However, this ratio may change quickly even several times a day but principally, the sympathetically overloaded athletes orient toward shorter racing distances requiring higher speeds while the parasympathetically overloaded athletes tend to perform longer race distances requiring higher endurance.

In the field of execution of trainings, the daily variability of the autonomic nervous system, as well as, the slow drift to any direction represent problems. The daily variance causes deviating responses of the athlete to the training practices. In case of a shift to sympathetic direction, the athlete warms up slowly, cannot perform the endurance exercises at a sufficient level, regeneration between the partial distances will not be efficient because the time required for regeneration increases. In case of a parasympathetic shift, the athlete slows down, does not tolerate the changes of intensity and the achievable maximum speed and pulse number decrease. If the coach does not accommodate to the changed nervous system conditions, the efficacy of the training is lacking, in worst cases it is harmful and may destroy capacities.

The daily, “fast” change of the autonomic nervous system influences the execution of training tasks but the improper selection of training types and the continuous forcing thereof react on the autonomic nervous system, which may affect the slow drift of the sympathetic-parasympathetic ratio. The inherited parasympathetic overload and the over-forced development of aerobic power may lead to parasympathetic overtraining. This means increase of parasympathetic overload and that the athlete is incapable of entering high intensity and pulse ranges, thus, “becomes obtuse”. The other extreme case when the basically sympathetically overloaded athlete performs proportionally too many anaerobic tasks so that the development of the aerobic capacity is depressed then sympathetic over training may occur, which causes unreasonable pulse changes, inefficient regeneration, continuous fatigue and depressive mood. Both overtrained statuses can be terminated with time consuming, special work lasting for several weeks.

Thus, the problem is twofold, on the one hand the daily changes of the nervous system significantly influence the performability of daily trainings and on the other hand the non-proportional way of skill development of trainings may cause the drifts of the autonomic nervous system. The problem is significant because due to the errors of the daily trainings, there is a lack in the targeted development of skills but in worst cases, it may even be destructive and so far there has been no method to quickly identify this situation. The other problem is that due to the sequentially wrongly selected training types, the training effects gradually lead to overtraining and when this is recognised, it is already too late, it is impossible to quickly modify the mistaken procedure.

Hungarian Patent Application No. P9403132 discloses an instrument measuring the limit of aerobic-anaerobic working based on blood flow that, if worn during sports activities, indicates the anaerobic metabolic activity in which the instrument's sensory and transforming stages are connected to micro controller units containing a signal processing program. The equipment according to P9403132 traces the peripheral changes of blood flow by the continuous monitoring of skin temperature and from this it determines the aerobic-anaerobic character of work done and provides a continuous diagnosis. By the immediate indication of the anaerobic threshold it protects the user from the harmful health effects of overload under anaerobic conditions. Though this instrument measures the skin temperature, it is suitable only for the alarming of the start up of the anaerobic energy producing system, but is not suitable for the determination of the capacity or the ratio between the two energy producing systems. At the same time, the value indicating the achievement of the anaerobic threshold is not defined in the description. How the anaerobic threshold is determined is not mentioned either.

WO 2014/102804 describes a medical diagnostics system for the diagnosis of Left Venticular Dysfunction (LVD). Though during diagnosis the skin temperature of the body is measured to indicate the activity of the body, this method is useful only to identify a pathological state and is not appropriate to monitor and assess the training efficiency of healthy athlete, especially not of the aerobic-anaerobic ratio.

During our research we recognised the fact that by measuring the skin temperature at points selected depending on the sports discipline and sport specific performance data, a specific method and a mobile instrument can be developed that is capable of monitoring and indicating the ratio of the aerobic and anaerobic energy producing systems and the operating of the sympathetic and parasympathetic nervous systems in order so that knowing the calculated data one could follow up the efficacy of training types and/or to elaborate a more efficient training program.

Accordingly, the invention provides a method for the continuous real time monitoring of the aerobic and anaerobic energy-producing systems and/or the status of autonomic nervous system, comprising: measuring the skin temperature of the body by a temperature sensor, and indicating the momentary intensity of the aerobic energy-producing system, the momentary ratio of the operation of the aerobic/anaerobic energy-producing systems and/or the momentary ratio of the sympathetic/parasympathetic power of autonomic nervous system based on the analysis of the temperature data; wherein the analysis includes the calculation of the gradient of temperature change in time that characterizes the ratio of operation of the aerobic and anaerobic energy-producing systems and/or the momentary ratio of the power of the sympathetic and parasympathetic autonomic nervous system.

In a preferred embodiment, the sensitivity of the temperature sensor is better than 0.1° C.

In another preferred embodiment, the indication is carried out in an audio and/or visual and/or a vibration manner.

In still another preferred embodiment, the monitoring is executed during sporting.

In still another preferred embodiment, the method further comprises the measurement of performance intensity data characteristic to the activity, such as force, acceleration, power and speed.

In still another preferred embodiment, the method further comprises the measurement of other functional data of the body, such as the pulse rate.

In still another preferred embodiment, the method further comprises the measurement of the transfer of processed data to a computer for processing, assessment and/or further analysis, optionally by a telemetric means.

In still another preferred embodiment, the skin temperature of the body is measured at several points on the body surface above the great muscles characteristic of the activity.

In still another preferred embodiment, method is carried out on humans.

In still another preferred embodiment,

a) the sporting activity is running and monitoring involves the measurement of skin temperature, speed of forward motion and optionally the pulse rate of the runner;

b) the sporting activity is paddling and monitoring involves the measurement of skin temperature, power output by the paddle and optionally the pulse rate of the runner;

c) the sporting activity is cycling and monitoring involves the measurement of skin temperature, power output and optionally the pulse rate of the runner;

d) the sporting activity is swimming and monitoring involves the measurement of skin temperature, speed of forward motion and optionally the pulse rate of the runner;

e) the sporting activity is team sport and monitoring involves the measurement of skin temperature, the sum of average acceleration in unit time in the X, Y and Z directions and optionally the pulse rate of the runner.

In still another preferred embodiment, the method is carried out on an animal.

In still another preferred embodiment, the animal is a racing horse.

By the analysis of skin temperature reaction changes due to exercise intensity changes, the invention enables the examination of the ratio of the aerobic and anaerobic energy producing systems and the current background of the autonomic nervous system. The indication of strong sympathetic tone is that for sudden intensity increase the skin temperature decrease is quick and steep, while for abrupt decrease to lower than AT value intensity the skin temperature increase starts very slowly even after 4 to 5 minutes. For strong parasympathetic tone at an intensity significantly higher than AT the skin temperature changes proportionally to the sudden intensity changes, i.e. the skin temperature increases with increasing intensity and decreases with decreasing intensity. The athlete and the coach even during warm-up, may notice the diverting nervous system reactions and if necessary may immediately modify the training protocol, as well as, from training to training it can be seen if the skin temperature responses to intensity changes forecast overtraining.

When testing the invention, apart from human measurements, the skin reflex changes was tested for animals as well. We tested race horses like Arabic thoroughbred at trainings for several months. Practically, the phenomena were identical to human experiences with a difference of the overload of the sympathetic nervous system that caused a greater degree of skin temperature decrease, i.e. anaerobic type reflex with the sudden increase of running speed. This difference may arise from the fact that the horse is a running-fleeing animal and evolution was beneficial to those individuals that were capable of escaping from the hunting animals with high speed even when “cold” during grazing. That is to say, they were rapidly able for high-level anaerobic energy production. This typical over-reaction was observable when the horse got frightened and got “prepared” for fleeing or during training when it heard the sound of another horse's galloping, i.e. if “the other runs then one must run”.

It was a general experience that for the Arabic thoroughbred it was difficult to achieve the energy producing system to work at high aerobic level (continuous, high intensity work) but when reached, the anaerobic reflexes significantly decreased, the effects of the outside world on energy production decreased, the dynamics of anaerobic overloaded reactions decreased and the endurance work became more effective.

### DETAILED DESCRIPTION OF THE INVENTION

We developed a sport physiological measuring system and method that is capable of the online monitoring of the ratio of the aerobic and anaerobic energy production system and the ratio of operation of the sympathetic and parasympathetic nervous system. The efficacy of training can continuously be controlled during training and if necessary by changing the original protocol (intensity, resting time, repetition characteristics) the training effect can be improved. The developed method may be applied in any of the sport disciplines where the ratio of the aerobic and anaerobic energy production systems is a significant parameter of sport performance.

The essence of the invention is the measurement of skin temperature on the skin surface preferably above the great primary or secondary muscles by special temperature sensor(s) depending on the sport disciplines whereby from the changes of what the momentary ratio of the aerobic and anaerobic energy production systems are well traceable and one can deduct the operating ratio and nature of the sympathetic and parasympathetic nervous systems.

For continuous loads under the AT (LT) intensities, the warm-up process starts within a few minutes both for less trained athlete or not trained people (with weak aerobic system) or for well trained athlete (with strong aerobic system), i.e. the aerobic energy production becomes dominant and this causes a temperature increase that is measurable on the skin surface. After warm-up, due to gradually increasing load, an increasing skin temperature can be measured for untrained and well-trained athlete alike, up to the intensity relating to the AT (LT) level. However, above the AT (LT) level the skin temperature of the weakly trained athlete starts to decrease, while that of the well-trained athlete increases further on. The better trained the athlete is (the stronger the aerobic system is), the higher the intensity where the skin temperature starts to decrease is.

Based on the analysis of the measured training sessions, it can be established that in the case of a well-trained athlete (having a strong aerobic system), the change of skin temperature above AT (LT) level shows the same increase and decrease profiles in low anaerobic (anaerobic extensive) range as in the aerobic range. This means that with increasing intensity the skin temperature increases and with decreasing intensity the skin temperature decreases. This changes when in the anaerobic range the intensity is so high (anaerobic intensive) that the anaerobic system suppresses the aerobic one, the efficiency of the aerobic system decreases and parallel to this the skin temperature decreases. This phenomenon was checked with lactate measurements and was established that above the AT (LT) levels after the intensity changes the change of skin temperature, i.e. the gradient characterises the momentary ratio of aerobic/anaerobic energy production systems. After increasing the intensity, for a positive gradient the aerobic system is dominating, and for a negative gradient the anaerobic system is dominating and when the gradient is zero the aerobic/anaerobic energy production systems are in balance, this being the maximum intensity when the athlete is able to work stably and continuously. The change of the gradient of the skin temperature indicates the change of the ratio of the aerobic/anaerobic energy production systems. The dynamics of the increase and decrease of temperature shows the disproportion of aerobic and anaerobic systems of the athlete. Temperature increase is steeper when the aerobic/anaerobic energy production ratio is shifted to the aerobic direction. Temperature increase decreases when the anaerobic energy production is more intense, as well as, the skin temperature decreases when the anaerobic energy production dominates and the temperature decrease is even steeper with more anaerobic energy production.

In case of sympathetic overtraining or the dominance of the sympathetic nervous system, even at intensities lower than the AT (LT) intensity level the skin temperature shows anaerobic type reflex, i.e. the increase of intensity is responded by a temperature decrease indicating that even the warm-up intensity level means a problem and the athlete cannot warm up properly. The athlete can work at high intensity for a short time but is incapable of performing endurance tasks.

In case of parasympathetic overtraining or the dominance of the parasympathetic nervous system, anaerobic reflex does not show up at all or only shows up for a very short time in the vicinity of maximum intensity. When the skin temperature decreases after increasing the intensity and the pulse rate increases, then it lasts for a short time (20 to 40 seconds) and due to a strong parasympathetic reflex the skin temperature rises again, the pulse decreases and the intensity drops down.

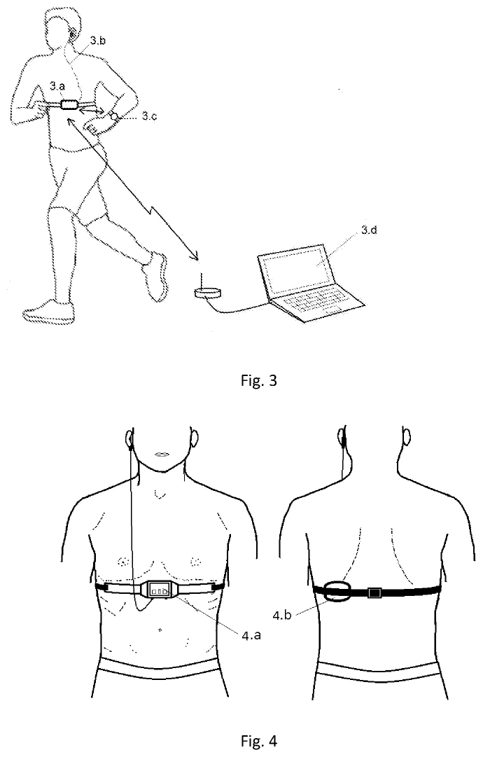

The measuring system shown in FIG. 3 includes a 3.a chest strap unit equipped with a temperature sensor, the 3.b earphone, the 3.c watch with LCD display and GPS and in certain cases a 3.d data processing, monitoring computer. The 3.a central unit is beneficially applicable for running sports connected to a chest strap containing pulse-counting electrodes. The task of the 3.a central unit is to process, store and transfer information of received and/or generated telemetric data. The 3.c watch with GPS measures the speed of the athlete and indicates the GPS position. The 3.a central unit is capable of measuring the pulse rate of the athlete, with the connected special temperature sensor it measures (on the surface of the back muscle of) the athlete's skin temperature, telemetrically communicates with the 3.d computer, calculates the physiological and technical data, transfers these data to the GPS-equipped LCD watch and via the 3.b earphone it verbally speaks the adjusted parameters. The adjusted parameters are also shown on the GPS-equipped LCD watch. The 3.d computer can telemetrically connect to the 3.a central unit and may request the stored data, as well as, it is able to online monitor the selected parameters. During training the athlete via the earphone and/or the LCD of the watch continuously receives the information that are required for the improvement of the factual skill, the coach may continuously check the training data on the 3.d computer, thus, the efficacy of training is much higher than without is.

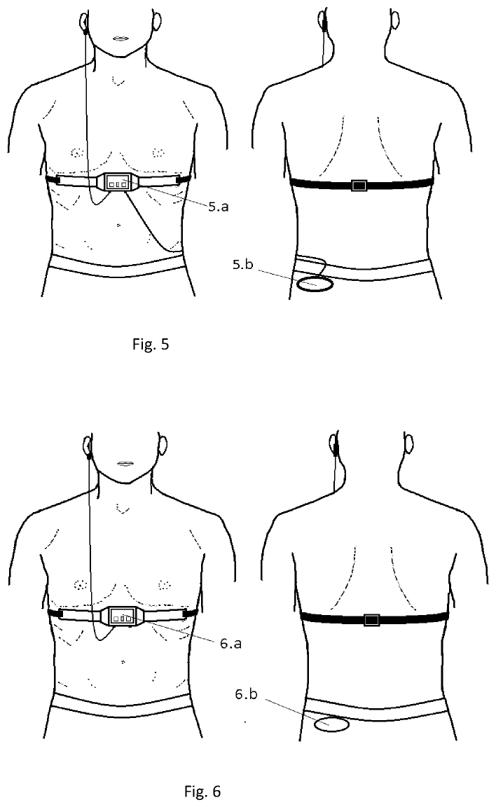

FIG. 4 shows the beneficial arrangement of the 4.b temperature sensor attached to the 4.a chest strap and the chest strap pushes the sensor to the back muscle of the athlete.

FIG. 5 shows the beneficial arrangement of the 5.b temperature sensor not attached to the 5.a chest strap but positioned onto the gluteus, connected by a cable.

FIG. 6 shows the beneficial arrangement of the 6.b temperature sensor that is not attached to the 6.a chest strap but it operates with an independent telemetric unit and transferring the temperature values to the 6.a central unit.

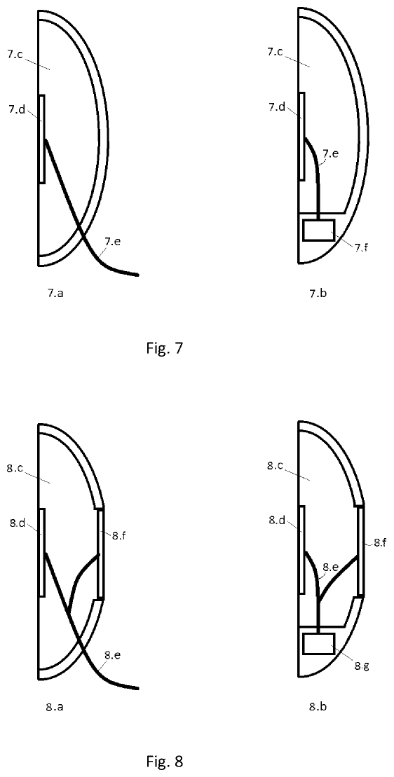

FIG. 7 shows the 7.a and 7.b temperature sensors. The 7.a temperature sensor is connected to the 7.b central unit by the 7.e cable and the 7.f telemetric module of the 7.b telemetric sensor transmits the temperature data to the central unit. The 7.d sensor of low thermal load reluctance is able to trace the small temperature changes and facilitates to measure the skin temperature with an accuracy of 0.01° C., thus the small changes are well observable. Because of the thermal insulation of the 7.c, these sensors are sensitive to then rapid temperature changes of the skin, however, are less sensitive to the changes of ambient temperature. The arrangement of the temperature sensor shown in FIG. 7 facilitates the positioning of it onto the great active muscles in the specific disciplines of sports.

In FIG. 8, the 8.a and 8.b temperature sensors contain two temperature measuring electronics of which one (8.d) adheres to the skin measuring the temperature of the skin surface and the other (8.f) measures the outer, i.e. ambient temperature. The double temperature measuring sensors are required when the environmental conditions, (e.g. strong, radiating sunlight, cold wind, cold rain) may influence the temperature of the skin by heating or cooling the measured surface. In this case, we can measure the direction and magnitude of heat flow and can disclose the erroneous consequences to the characteristics of the energy producing processes. The 8.a sensor connects to the central unit via cable 8.e and the 8.b telemetric sensor's 8.g telemetry module transmits the temperature data to the central unit. The 8.d and 8.f sensors of low thermal reluctance are capable of tracing rapid temperature changes and facilitate to measure the changes of the skin's and ambient temperature with an accuracy of 0.01 C, thus even the small changes are well observable. The 8.c. sensors are sensitive because of their good thermal insulation, but are less sensitive to the changes of the ambient temperature. The arrangement of the temperature sensor shown in FIG. 8 facilitates the positioning to sports branch specific active, great muscles.

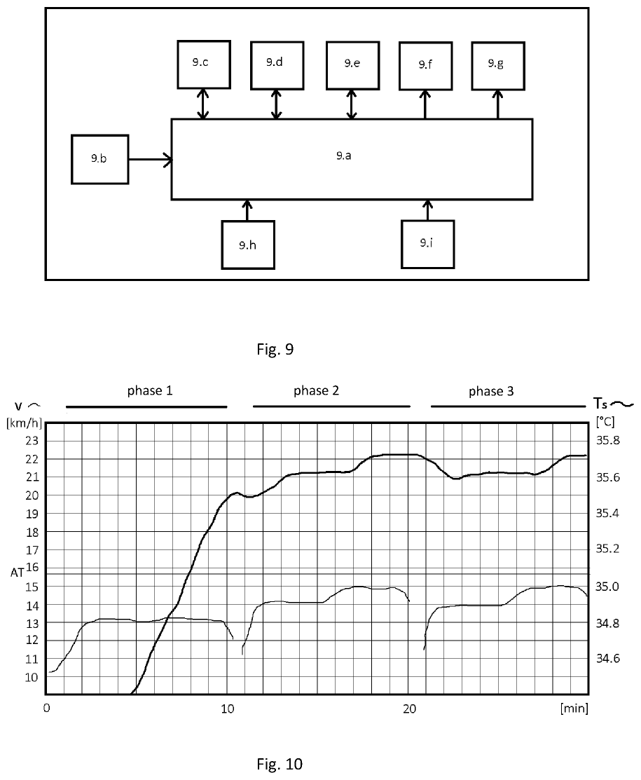

FIG. 9 shows the block diagram of the 3.a central unit. The unit according to the invention contains the 9.a data processing block, the 9.b data-collecting block, the 9.c telemetric, communication block, the 9.d data storage block, the 9.e computer communication block, the 9.f display block, the 9.g audio block, the 9.h energy supply block and the 9.i input block (pushbuttons).

The measuring system shown in FIG. 3 is capable of the online measurement of sports physiological and technical parameters. It continuously measures the speed, the pulse rate of the athlete, the skin temperature sensor measures the skin temperature of the runner athlete and if necessary, the ambient temperature. The skin temperature is measured with an accuracy of 0.01° C. to compare it with the athlete running speed as sporting intensity should be suitable for the continuous monitoring of the status of aerobic and anaerobic energy producing systems. The ambient temperature is required to be measured when the environmental thermal effects influence the accuracy of measurements and it is demanded to indicate this modifying effect. The continuous monitoring of energy producing systems and the continuous communication of the information characterising the status of the energy producing system are novel and unique in their nature that help the athlete during the training to know the status and ratio of the operation of the energy producing systems. So far, there has been no laboratory or mobile equipment suitable for this purpose.

During the practice of the various types of trainings it is well observable that as long as the aerobic system is dominating a higher temperature is relevant to the exercises of higher speeds and lower temperatures are relevant to lower speeds, i.e. the skin temperature increases or decreases with the increase or decrease of the running performance. However, in case of anaerobic dominance, a decreasing temperature relates to increasing speed, as well as, above a certain speed the temperature continuously decreases and does not reach equilibrium.

In FIG. 10, the thin line represents the speed (v) of the runner in km/h, the thick one represents the skin temperature of the athlete (Ts) in ° C. AT=15.7 km/h indicates the anaerobic threshold, i.e. above 15.7 km/h the anaerobic energy producing system dominates. The figure shows 3 phases of training. Phase 1 is the warm-up, phases 2 and 3 are identical, the task was to run at 14 km/for 4 min and at 15 km/h for another 4 min. It may well be seen that all three phases were in the aerobic zone, in phases 2 and 3 the change of temperature with a small delay but closely follows the change of intensity.

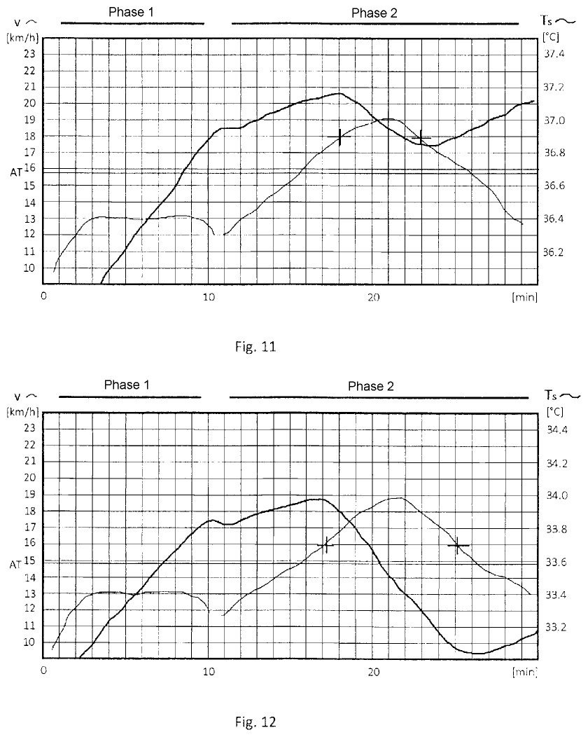

In FIG. 11, 2 phases of training are shown, Phase 1 is warm-up and Phase 2 is gradual increase then decrease of intensity. The task was to increase the running speed from 12 to 19 km/h in 10 min after warm-up then decrease to 12 km/h. In this period of time the athlete had a strong aerobic system, he started to cool down well above the anaerobic threshold (AT=15.7 km/h), at 18 km/h, i.e. till then the aerobic system dominated but here switched and the anaerobic system became overwhelming. When the speed fell below 18 km/h again the aerobic system became dominant, the cooling of the skin surface decreased and after 1 min the temperature started rising, again.

FIG. 12 shows the same training task as in FIG. 11, however, in this period due to strong anaerobic training the anaerobic capacity of the athlete strengthened against the aerobic one. It may well be seen that the anaerobic system became stronger in Phase 2 at about 16 km/h, the athlete started cooling and keeps on cooling until the speed decreases again below 16 km/h.

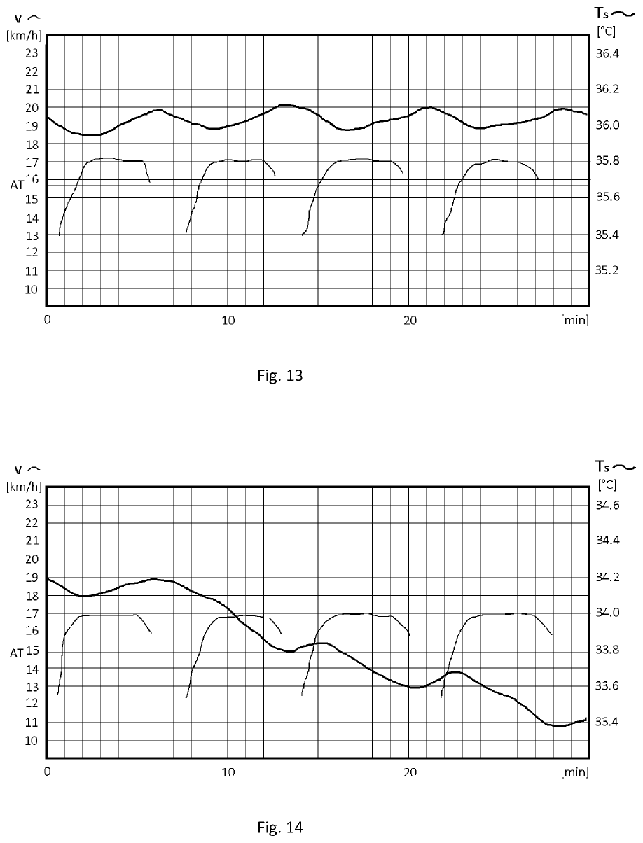

FIG. 13 shows an interval training. The task was to reach the speed of 17 km/h in 1 min and keep this for 3 min then there was a 1.5-2 min rest. In this period of time the aerobic system of the athlete was strong and was able to do training in the anaerobic zone, too, with overwhelming aerobic system, i.e. the anaerobic system did not suppress the aerobic one, the athlete cooled down in the resting phases, but in every interval the athlete warmed up to the temperature correlating to the speed of 17 km/h. The training shown in FIG. 13 improves the aerobic system, increases aerobic power.

The training task shown in FIG. 14 was identical to the one shown in FIG. 13, however, the aerobic capacity of the athlete was lower due to the previous stronger anaerobic trainings. It may well be seen that after the first section the aerobic system became tired, at the speed of 17 km/h the athlete continuously cooled, i.e. the anaerobic system was dominant. It should be noted that the trainings shown in FIGS. 13 and 14, apart from the fatigue feeling and the skin temperature parameters but based on the traditionally measured parameters appear to be completely identical (speeds, pulse responses, partial times), however, monitoring the skin temperature objectively showed that the training effects and the metabolic ratios and processes were completely different. The training shown in FIG. 14 destroys the aerobic system, lowers the aerobic power. When the athlete and/or the coach receives information about the running speed, the absolute values of skin temperature and the momentary gradient, then it becomes unambiguous that according to the momentary status the intensity should be decreased in order to avoid the destroying effect.

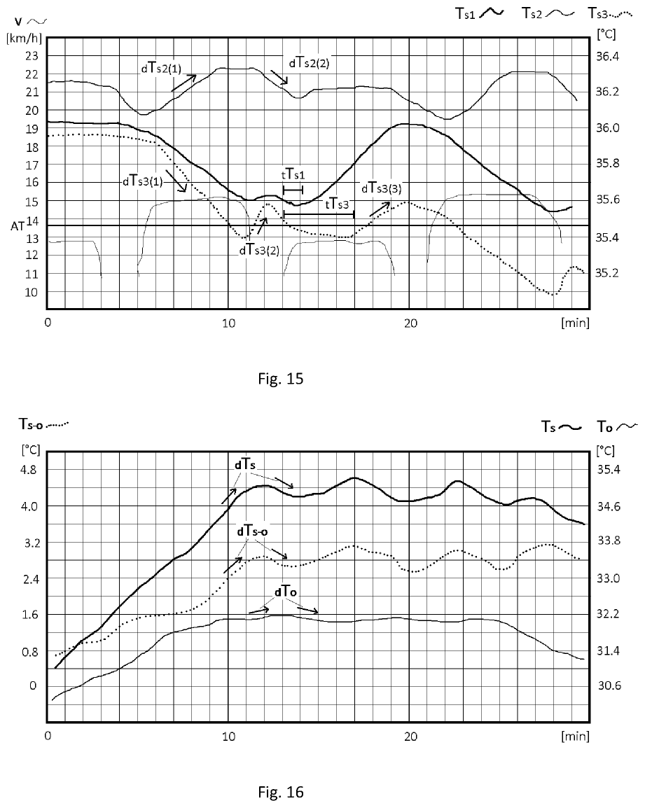

FIG. 15 shows typical skin temperature curves for the various sympathetic/parasympathetic ratios of the autonomic nervous system. After warm-up the runner runs for 6-minute periods with 1-1.5 km/h above and below the AT level with 2-minute rests.

For the sympathetic/parasympathetic ratio in equilibrium the skin temperature of the athlete behaves according to Ts1. The athlete responds to the intensity changes quickly, within 1-1.5 min. The skin temperature increases below the AT level and decreases above AT speeds.

For parasympathetic overtraining or significant parasympathetic nervous system dominance at speed above AT the skin temperature increases according to the aerobic overloaded reflex (dTs2(1)), when the speed decreases the skin temperature decreases rapidly (dTs2(2)), which also represents an aerobic overloaded reflex status. The increase of parasympathetic overload is indicated by the fact that the athlete only at increasingly higher (above AT) speeds and for shorter periods of time is able to respond with the anaerobic reflex, hence with the decrease of skin temperature.

Ts3 skin temperature curve indicates sympathetic over training or significant sympathetic nervous system dominance After warm-up, when the athlete reaches above the AT level speed, his/her skin temperature quickly and almost immediately responds with anaerobic overloaded reflexes, i.e. with the strong decrease of skin temperature (dTs3(1)). When lowering the speed, as a response of the suddenly disappearing anaerobic overload, short, strong increase of skin temperature is experienced (dTs3(2)), that lasts for 1-1.5 min. It is typical of the sympathetic overloaded status that for relatively small, below AT speeds the athlete responds very slowly with aerobic reflex. Instead of the normal 1-1.5 min (tTs1), 4-5 min (tT3s) may elapse until the increase of the skin temperature (dTs3(3)) indicates the aerobic reflex. Due to the quick anaerobic and the slow aerobic reflexes in sympathetically overloaded status and after warm-up, the efficacy of the appropriately operating aerobic system gradually decreases, which has a destroying effect on the aerobic power and may lead to the loss of competitiveness and trainability.

During exercise, the ratio and efficiency of the two metabolic energy-producing systems change continuously. The value of the relatively well measurable AT (LT) greatly depends on the momentary status of the athlete and the environmental threshold conditions and may change within a training period. As the value of AT changes, so does the operating intensity of the aerobic and anaerobic system change when the external conditions influencing the motion change Such conditions are for cycling and running: upwind, downwind, slope, or for kayaking, rowing: upwind, downwind, waves, as well as, for any sports: ambient temperature and Sun radiation. When within a training event the external conditions (ambient temperature, up- or downwind, dressing, undressing) influencing the skin temperature of the athlete do not or hardly change then the absolute temperature well indicates the operating intensity of the aerobic system and the temperature gradient indicates the ratio of the aerobic/anaerobic energy-producing systems. The examples given by FIGS. 10 to 15 all suppose laboratory conditions, i.e. the external conditions are unchanged during training. For trainings using running machine, roller bicycle or kayak ergometer the absolute value of the skin temperatures, as well as, the gradients of changes when measured for a certain athlete and a defined point can be compared from training to training. The absolute temperature should be determined that indicates proper warm-up, i.e. the required aerobic energy-producing intensity, as well as, those temperature decrease gradients belonging to the anaerobic loads to accurately execute the zone trainings Under laboratory conditions, the absolute skin temperature values related to a body surface point well characterise the intensity of the aerobic energy production for a certain athlete. When the training or competition of the athlete is under extreme weather conditions (strong sunlight, high or cold ambient temperature, rain, cool wind) the double sensor system should be used as shown in FIG. 8 in order to obtain accurately measured data.

It is important to deduce the real changes of temperature from the data obtained from thermometers smoothed to the skin surface, excluding the external effects influencing the sensor. The sensor shown in FIG. 8 is equipped with an external thermometer (8.f) measuring the temperature under the dress and the internal thermometer (8.d) measures the skin temperature. When e.g. the athlete wears a black item of clothing above the sensor then due to strong sunlight the outer surface of the garment may reach a temperature of 45 to 50° C. because of which the sensor may warm up together with the garment and this may cause the warming up of the skin surface. It has a contrary effect when the athlete is exposed to e.g. cold rain or wind, the garment itself cools down and may cool down the sensor and the skin as well. The measurement will indicate the skin cooling down that is not true. One should be alert to the measurement circumstances due the 0.01° C. accuracy of the temperature measurement.

With the double-thermometer system one can measure the direction of heat-flow because the heat-flow always tends to reach equilibrium, i.e. the higher temperature medium heat up the lower temperature one or in other words, the lower temperature one cools the warmer one. Using the double thermometer system it can accurately be measured if the skin warm up or cools the garment smoothed to the skin surface or vice versa.

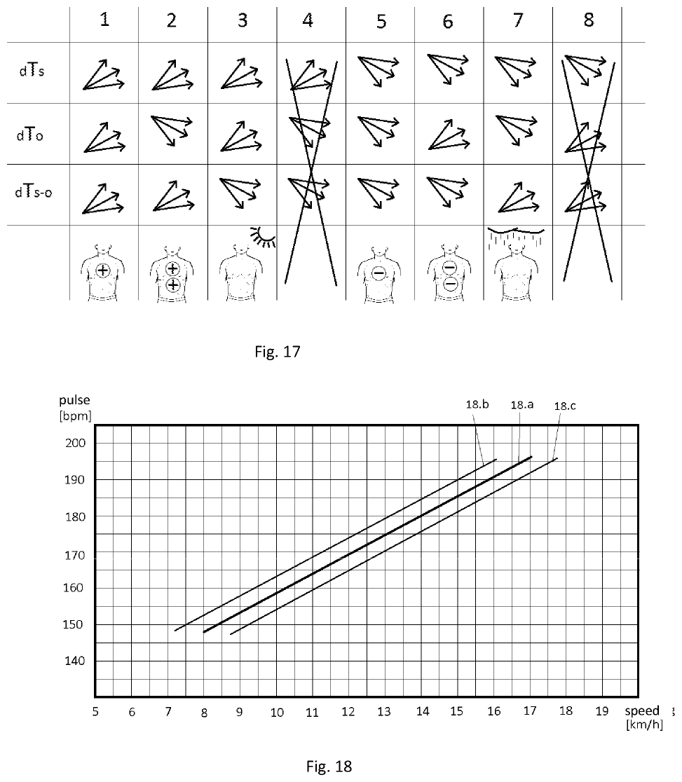

In FIG. 16, Ts (Tskin) indicates the measured skin temperature, To (Tout) shows the temperature of the external surface of the sensor below the garment. Ts-o=Ts−To indicates the temperature difference between the skin surface and the external surface of the sensor. dTs indicates the change of the skin surface, i.e. the gradient, dTo is the gradient of the external temperature under the garment and dTs-o denotes the gradient of the difference of the skin and external temperatures. Using the double sensor system on can conclude the type of heat-flow whether the skin is dominating or the external effects. When skin is dominating then it warms or cools the garment, otherwise vice versa, the skin is warmed or cooled via the garment. The various possibilities are summarized in the Table of FIG. 17.

The Table of FIG. 17 shows the possible variants of the change of the gradients of temperature curves of FIG. 16. The arrows pointed upwards at different angles indicate the positive gradient the ones pointed downward indicate the negative ones.

In column 1, the skin temperature (Ts) and the external temperature (To) are both increasing. Since Ts-o (=Ts−To) also increases this means that the skin temperature increases more than the external one, i.e. skin temperature is dominant warming the air under the garment and the garment. This phenomenon indicates the increase of aerobic power.

In column 2, despite the decrease of the external temperature the skin temperature rises, i.e. the skin shows strong warm-up so that even the environment cannot cool it down. The phenomenon indicates strong aerobic overload.

In column 3, the skin temperature and the external temperature under the garment increase but the dTs-o gradient is negative, i.e. the external temperature increases more than the skin temperature. This means that due to the external thermal effect the skin is warming, e.g. in case of strong sunlight.

In column 4, the visible variant does not exist. When the skin temperature increases and the external one is decreasing then Ts-o (=Ts−To) should not decrease.

In column 5, the skin temperature decreases and the external temperature decreases. Since Ts-o also decreases this means that the temperature of the skin decreases more than the external one, thus the skin is the dominant one that cools down the air under the garment and the garment. This phenomenon indicates an anaerobic overloaded reflex.

In column 6, despite the increasing external temperature the skin temperature still decreases, i.e. skin shows such a strong cooling that even the environment cannot warm it up. This phenomenon indicates a strong sympathetic reflex and strong anaerobic power overload.

In column 7, the skin temperature and the external one under the garment, both decrease but the dTs-o gradient is positive, i.e. the external temperature decreases more than the skin temperature. This means that due to the external effects the skin cools, e.g. due to rain or cool wind.

The variant in column 8 does not exist when the skin temperature decreases and the external one increases, then Ts-o (=Ts−To) cannot increase.

Besides the characteristic states shown in FIG. 17, the analysis of Ts-o temperature difference data can help to decide whether the garment of the athlete is sufficient for the sporting activity. When the Ts-o value approaches 0° C. or perhaps falls below, then it is likely that the athlete is overdressed but when Ts-o is high compared to the usual (Ts>>To), the athlete is rather underdressed. To be underdressed means a problem because the athlete cannot warm up optimally and in extreme cases due to feeling cold such a high intensity warm-up is required that it means anaerobic zone overloading and the warm-up, i.e. the achievement of the effective operation of the anaerobic system is impossible. For being overdressed, apart from feeling uncomfortable, the athlete becomes overheated even during warm-up and due to the increased feeling of warmth warm-up will not be appropriate, the athlete cannot work at high intensity, also sweating increases that causes rapid dehydration. Based on our experiences, the background of the phenomena should always be analysed, as a swimmer is always in water at around 26-27° C. which represents quasi “laboratory” conditions, while a cyclist for example has to fight the extreme weather conditions. For the external temperature of 15 to 25° C., when wearing optimal sports dresses the measurable Ts-o values are 3 to 5° C. for running, cycling and kayak-canoeing. The difference of below 2° C. relates to overdressing and that above 6° C. relates to under dressing.

The skin temperature change indicating the operation of the metabolic energy-producing system varied depending on the individual, the sport discipline and the muscle activity and is of differing character and degree at different points of the body surface. Prior to the regular use of the sensor the optimal measuring point or points should be selected on the skin surface. It is preferable to examine the test measurements in parallel with sensor on the primary or secondary muscle at several points. For ergometric testing of cycling, the sensors placed onto the calf of leg, thigh muscle and gluteus show temperature changes varying in time and dynamics After starting the exercise the redistribution of the cardiovascular system starts more efficiently at the primary muscle groups more intensively used, there, the aerobic energy production starts faster, the warming up occurs earlier, due to the measurable temperature rising the skin reflex starts earlier and shows a steeper gradient. When gradually increasing the intensity, in case of the weaker or more used secondary muscle groups the energy production first changes to anaerobic, which in case of anaerobic overload, with respect to other muscle groups results in a decreasing skin temperature reflex that is measurable earlier.

The optimal sensor position should be selected according to the skill improving strategy. If one wants to check the warming up process, then the “strongest” (slowest) point should be selected on the primary muscle. For the check of the anaerobic overload, the “fastest” (the weakest) point should be monitored on the secondary muscle. We may get a more accurate picture of the momentary status of energy production in the various muscle groups when using several sensors in parallel regularly. For kayaking that uses almost the complete muscle tone, it is indifferent whether the leg muscle, arm muscle or the back muscle starts stiffening (high degree of anaerobic overload, spasmic pain), the efficiency of kayaking falls suddenly. The multi sensor measurement is capable of mapping the athlete to find what muscle group is the weakest to achieve the target, i.e. which one should be developed the most intensively. During the improvement of skills one should be careful to compare identical position measurements based on the skin temperature reflex.

The measurement of skin temperature indicating the operation of the metabolic energy-producing system should be examined at a selected point, using a sensor located on the surface of the skin as the function of the power output of the athlete. In sport disciplines where the activity ratio of the various muscle groups is different within a training program (e.g. triathlon, swimming, athletics) it is preferable to use several sensors.

In sports, instead of power output that is difficult to measure, power type parameters may be measured that well characterise the intensity of motion, the intensity of sporting activities. It is important to perform reproducible measurements and to measure the parameter ever more accurately, and it “should be characteristic of the sport”. Usually, the AT value is correlated to various parameters depending the sport disciplines, e.g. the (horizontal) speed for swimming and running, average thrust of the paddle for kayaking and rowing, power output for cycling expressed in W.

For the sports where the weather conditions may influence the operation of the metabolic energy-producing system the double-sensor system may improve the accuracy of the measurement and the influence of external effects becomes measurable.

Depending on the sports an aerobic/anaerobic operation ratios correlate to the various intensities, which can be changed with training. The optimal aerobic/anaerobic ratio must be selected according to the racing distance/duration of the athlete. If a certain racing distance is covered primarily in aerobic metabolism then the athlete will easily endure the race but might be slow. On the contrary, when the anaerobic load is overwhelming for a certain racing distance then the athlete will be faster, however, may not last till the end of the distance. Therefore, during the trainings the aerobic/anaerobic ratio shall be adjusted so as to facilitate the endurance of the whole distance at the highest possible speed. For a well trained athlete concentrating on a certain racing distance will be the best because a certain aerobic/anaerobic ratio is optimal for only one racing distance.

Conconi test is a basic sports physiological test. First, it was developed to test runners at a 400-meter path, gradually increasing the speed while measuring the pulse rate. Thus, the output power (the motional intensity) of the athlete is characterised by speed. FIG. 18 shows the speed/pulse straight lines of the Conconi test. It can be seen that the pulse rate of the athlete changes proportionally to the running speed (18.a). Performing periodically the Conconi test, we can get an insight of the momentary status of the athlete; when the speed/pulse rate straight line shifts to the right (18.c), the a lower pulse rate corresponds to a defined speed, the athlete is more trained and is in a better condition. When the straight line shifts to the left (18.b), then a higher pulse rate corresponds to a defined speed, the athlete is less trained and is in a worse condition. Conconi test may be applied to other sports apart from running but the performance (kinetic intensity) of the athlete is substituted by other parameters such as the performance measured by a wattmeter built in a bicycle, the measurable paddle thrust per unit time in kayak-canoe, with speed in swimming Similar to other sports physiological tests, even Conconi test does not provide information on the ratio of operation of the aerobic and anaerobic systems at a particular intensity, however, this is essential because this indicates “how much” it takes from the athlete to keep up the particular intensity, like for seconds, minutes or hours. Utilizing the invention in a genuine sports environment, the aerobic/anaerobic ratio of energy production can be monitored continuously that provides a new, important, third dimension to the classical intensity/pulse rate response. For running, the skin temperature and the running speed shall be compared (FIGS. 10 to 15). In other sport disciplines, the parameter characteristic to the sport shall be measured: e.g. for swimming, the swimming speed, for cycling, the performance in W, in kayaking, the paddle thrust, for team sports (handball, basketball, soccer, hockey) the sum of the average acceleration of the body in unit time in the X, Y and Z directions.

When the typical performance type parameter characteristic to the particular sport is not or only hardly measurable, it can be substituted by the pulse rate of the athlete to a limited extent. In practice, within a training period, the pulse rate of the athlete changes linearly to the power output, i.e. the pulse rate and skin temperature data can well be used to control the load characteristics (optimal warm-up pulse rate, pulse rate related to the maximum aerobic power, pulse rate limit corresponding to anaerobic overload). Since the pulse rate response of the athlete to a particular performance depends on training and fatigue (18.b, 18.c) the pulse rate based data of analysis may significantly differ from the data of accurate performance based analysis within a week.

When during training, the procedure of skin temperature measurement is applied with an accuracy better than 0.1° C. and the athlete receives information about the value and momentary gradient of the skin temperature of the active muscles, there is the capability to control the aerobic and anaerobic energy production with the help of only the temperature parameters, i.e. by increasing or decreasing the intensity one can activate the demanded energy-producing process and can regulate the degree of energy production. By this, depending on the training task the athlete is capable of accurately developing the aerobic or anaerobic capacities to be improved. During development, we regularly tested the operating mode of the arrangement shown in FIG. 3 in which the athlete monitored the data deduced from the values of skin temperature and the momentary gradient thereof to control the equilibrium of aerobic and anaerobic energy production in high intensity training sections. This is essential for the preparation of top competitors whatever performance sports are considered. However, the sole skin temperature control can be used only during training because during training the athlete is completely aware of the fact whether he/she is resting or at what intensity he/she performs the task. The stored skin temperature data are useless for further processing without the power or pulse rate data because the cooling or warming procedures may be understood only as a function of the output intensity parameter characteristic of the of the different kind of sports or the pulse rate response.

For competitive athletes, it is very important to determine and develop the ratio of aerobic and anaerobic capacities corresponding to the racing distance. Since the development of aerobic and anaerobic capacities have effects on each other, the accurate training effects are achievable by monitoring the ratio of the activities of the two energy-producing systems. The control is more precise the closer is the feedback, i.e. the athlete or the coach immediately notices the unbeneficial disproportion and alters the course of training (intensity, rest, repetition number, etc.). Thus, it is important that the athlete and the coach has the possibility to continuously monitor the parameters that provide information on energy production. From the content of information point of view the sporting intensity and/or pulse rate response, the skin temperature above the primary or secondary muscle or the data deduced thereof, the momentary gradient of the skin temperature or the data deduced thereof and the conditions influencing the skin temperature (radiant heat, external cooling, under-dressing, overdressing) are of importance. It is useful for the athlete and the coach to query the 6 possible statuses shown in the lower line of FIG. 17 because by doing so the erroneous decision due to mistaken data can be eliminated. The fact that the skin “wants” to cool or warm up should be indicated to the coach and the athlete as accurately as possible. This principal parameter depending on the sports should be made measurable, as well as to select the optimal way of communication. This is easier under laboratory conditions, while in a sporting environment it is more difficult because corrective calculations have to be applied that are primarily defined by the sport and the environment thereof. It is advisable to introduce the “sX” (skin reflex) value relating to the absolute value of skin temperature and “dsX” (delta skin reflex) value relating to the change of skin temperature. When e.g. “sX” is defined as the absolute temperature—30° C., the athlete can be instructed to warm up until “sX” reaches the 5.5 value. Or e.g. for triathlon one has to distinguish between the critical anaerobic loading levels: “dsX_s” for swimming, “dsX_b” for cycling and “dsX_r” for running according to skin reflex.

There are various possibilities to communicate the important information depending on the sport, e.g. for running and kayaking, it is optimal to communicate verbally via an earphone, for cycling and LCD display fixed to the handle-bar, for swimming a vibrating motor will do, and for the coach, a tablet (computer) is the correct device. Storage, further processing and statistical analysis of the measured data help the strategic punctuation of preparation.

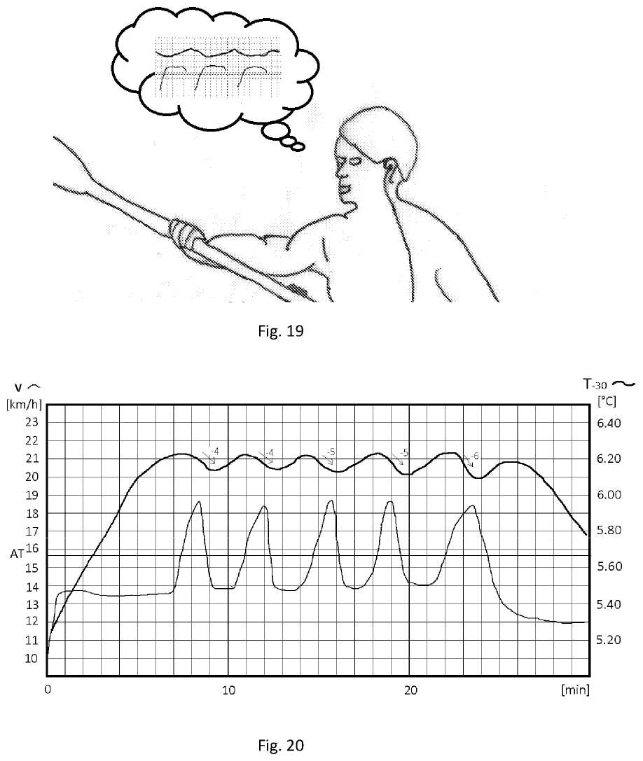

Accordingly, during training and races, the communication of information to the athlete may occur via any audio-visual display, black-and-white, colour, graphic or alpha-numerical display or LED display, as well as, using vibration or sound information that can be verbal or a special sound effect. The important thing is that the athlete should see or imagine the “usual curves” of intensity (running speed, cycling performance, paddling thrust), the actual aerobic power (absolute skin temperature or data calculated thereof) and the aerobic/anaerobic ratio (momentary gradient of skin temperature or data calculated thereof), (see: FIG. 19). If this takes place during the daily trainings the preparation of the athlete can be achieved significantly more safely and accurately than earlier without the help of the invention. The athlete is capable of controlling not only the intensity according to the prescribed target zone but is also capable of performing the tasks related to the metabolic energy-producing system (paying attention to the defined aerobic overload, to reach the prescribed anaerobic zone) that so far has not been achievable.

As an example, FIG. 20 illustrates the training of an endurance sport that is a short fartlek type training. The athlete adjusted the chest strap central unit so as to hear the running speed via the earphone with an accuracy of 0.1 km/h, the above 30° C. portion of skin temperature on the secondary muscle with an accuracy of 0.01° C. and the gradient of skin temperature that is always the current skin temperature, minus the skin temperature measured 15 sec earlier with an accuracy of 0.01° C., multiplied by 100. The data are announced via the earphone with 3 sec pauses: e.g. speed: 13.4; temperature: 5.47; gradient: −5. The training task is the following:

Warm-up: light run with a speed of 12 to 14 km/h until the temperature reaches 6.20.

Pace change: 15 min continuous running, the speed of the runner should be increased so that the gradient be less than −4 then light running until the gradient becomes positive and the skin temperature be above 6.20.

Cooling down: light running at 12 km/h for 10 min.

Even in the anaerobic range the training strengthened the runner so that his/her aerobic capacity was not shortened. The runner was fully aware of what intensity zone and with what aerobic/anaerobic ratio he/she was working. All way long the training fell into the aerobic overload except when he/she was running at high intensity with negative gradient. The resting time depended on the time needed for regeneration, i.e. his/her aerobic system could work at the prescribed intensity. During the training, the runner could control the duration of the optimal resting time depending on the feedback from the skin temperature data for the purpose of skill improvement that could not have been achieved without this invention.

As an example of a power endurance sport, FIG. 21 shows a leader-changing kayak training A power meter built into the paddle measures the intensity. The thinner line in FIG. 21 represents the average thrust output in unit time (aF) in kp. Via an earphone connected to the chest strap central unit the athlete could hear the average thrust with 0.1 kp accuracy; the portion of skin temperature above 30° C. on the secondary muscle with 0.01° C. accuracy; and the gradient of skin temperature that is always the current skin temperature, minus the skin temperature measured 15 sec earlier with an accuracy of 0.01° C., multiplied by 100. The data are announced via the earphone with 3 sec pauses: e.g. thrust: 7.4; temperature: 5.47; gradient: −5. The coach could see the above data from the motorboat escorting the kayakers. The training task is the following:

Warm-up: paddling with a thrust around 6 to 7 kp until the skin temperature exceeds 6.00.

Lead switching for 45 min: two kayakers paddle side-by-side leading one after the other. The kayaker in front dictates the pace the other one sits in the “downstream” which means that by lagging a little (2 to 3 meters) looks for a position where the water is continuously “sloping”, i.e. the nose of the kayak is always a little bit lower than the back. Thus the kayaker sitting in the “side water” paddles as if on a “slope” and therefore, can effort much less power than the leading one, can relax, regenerate and rest for the next lead. Kayakers “a” and “b” lead switching every 3 min during the 45-minute duration task. The intensity of the leading kayaker should be around 10 kp thrust.

Cooling down: light paddling at 6 kp for 10 min.

FIG. 21 illustrates the parameters of “a” kayaker. On the top of the Figure the leading kayaker (a-b-a-b- . . . ) is shown for the particular 3 min period. After the 2^{nd} switch kayaker “a” heard that the gradient of skin temperature remained negative even when he was not in leading position, therefore, notified the coach that there was a problem with the pace and cannot get regenerated. The coach saw on the tablet that the pace was too high for kayaker “a”, due to anaerobic overload the skin temperature continuously decreases even when not leading just “sitting on the downstream” and if they would carry on in the same way the aerobic system would be overloaded that could even reach the level of capacity decrease. Since this is an aerobic capacity improving training the coach told them to take back from the pace and the leader should remain at around 9 kp thrust. However, this also meant that the intensity of the kayaker behind decreased, as well, so when “sitting on the downstream” could regenerate, the anaerobic overload ceased and could warm back above 6.00. After little intervention the training achieved its skill improving target because the kayaker could work with high aerobic power, although there was an anaerobic overload during the 3-minute leads there was an opportunity for regeneration within the 3-minute rests.

Without monitoring the skin temperature, i.e. the continuous control of the aerobic/anaerobic ratios it would not have turned out even after the 2^{nd} exchange that there would be a problem with kayaker “a” and instead of an aerobic power improving training there could have been an anaerobic overloading training.

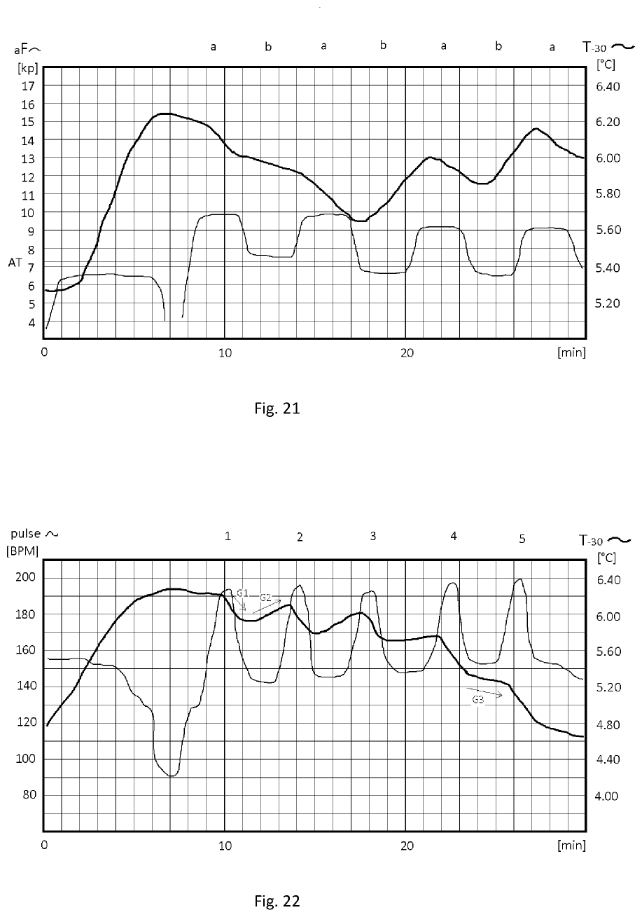

As an example, FIG. 22 illustrates a team sport training that is an ice-hockey technical training. The training was a match simulation practicing an offensive situation at an intensity corresponding to realistic loading and with rests. According to the protocol of a quarter the players were in offense for 1 min and had 3 min rest and they repeated this 5 times. During rest the players were not sitting on the bench but were lightly skating with relaxing character. The training was of technical type but the practice required maximum intensity that is typical of the ice-hockey sport. The chest strap central unit stored the 3D mass acceleration data and pulse rate and skin temperature data as sporting intensity data. Due to the character of the training there was no need for online information, the athletes had to focus on the technical tasks. The measured data were analysed afterwards from the viewpoint that to what extent the one-quarter training was loading the athlete.

The coach wanted to analyse the outcome of the aerobic capacity and the pulse relax from the fatigue feeling aspects. In FIG. 22 it can well be seen that warm-up was proper, the player started the game with high aerobic operation. At technical task 1 an extremely strong temperature decrease (G1) indicated a very high anaerobic overload and this eroded the aerobic system so much that regeneration was not sufficient even in the rest period and the skin temperature could not rise appropriately (G2). The player started the 2^{nd} task at a lower aerobic background and this tendency continued in the further cycles. After task 4, the fatigue reached such a level that even after the rest the skin temperature could not increase (G3), i.e. the aerobic system could not regenerate at all. The worse aerobic background of the player is indicated by the fact that the relax of the pulse rate was not sufficient, while after task 1 it could go back to 142 but after task 4 it fell back only to 154. Unfortunately, the player started playing inaccurately in task 3 and this became even worse in tasks 4 and 5.

It can unambiguously be seen that the player is not suitable to play matches, should perform improving trainings in order to be able to play even one quarter accurately.

By using the invention and analysing the trainings and matches one can forecast the “playing power” of the players, the skill improving trainings can be commenced in time, one does not have to wait for the formal decline to be indicated by ever more inaccurate play.

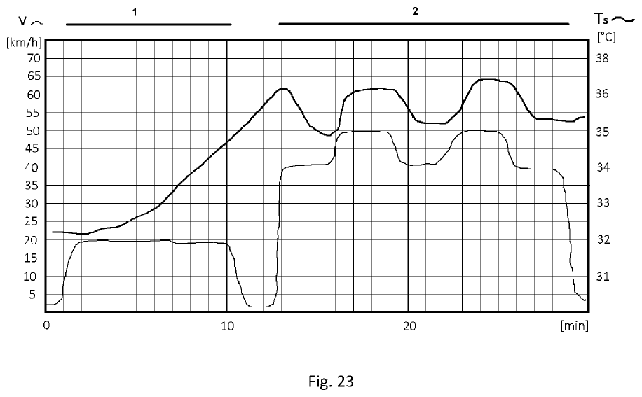

FIG. 23 illustrates the regenerating training of an Arabic thoroughbred horse. The telemetric thermal sensor was installed under the strap fixing the saddle, on the shaved skin. Section 1 of the training included a 10-minute warm-up trotting then after a short rest a 15-minute pace changing galloping (2). The jockey changed the speed every 3 minutes between 40 and 50 km/h. The “starting anaerobic reflex” so typical of the Arab thoroughbred can well be seen that is an always observable skin cooling when the horse starts galloping from trotting or standing still, i.e. within a few seconds the horse accelerates its 400 kg mass to 35 to 40 km/h. At the start of galloping the anaerobic skin reflex appears shortly, followed by the characteristic skin temperature changes of purely aerobic loading, i.e. it increases at higher intensity and decreases at lower ones.

1. Method for indicating the status of the aerobic and anaerobic energy-producing systems and/or the status of autonomic nervous system of a human or animal subject during sporting, comprising:
repeatedly measuring the skin surface temperature of the subject;
determining a temperature gradient of the skin temperature along time, based on the temperature data measured;
determining the momentary intensity of the aerobic energy-producing system, the momentary ratio of the operation of the aerobic/anaerobic energy-producing systems and/or the momentary ratio of the sympathetic/parasympathetic power of autonomic nervous system, based on the temperature gradient determined;
determining the status of the aerobic and anaerobic energy-producing systems and/or the status of the autonomic nervous system of the subject, based on the momentary intensity of the aerobic energy-producing system, the momentary ratio of the operation of the aerobic/anaerobic energy-producing systems and/or the momentary ratio of the sympathetic/parasympathetic power of autonomic nervous system determined;
generating a signal indicative of the status of the aerobic and anaerobic energy-producing systems and/or the status of autonomic nervous system of the subject; and
outputting the signal relating to the subject in a sensible form.

2. The method according to claim 1, wherein the temperature is measured on the body surface above a primary muscle characteristic of the sport.

3. The method according to claim 2, further comprising:
repeatedly measuring the temperature on the body surface above a secondary muscle characteristic of the sport;
determining a further temperature gradient of the further skin temperature along time for determining the momentary intensity of the aerobic energy-producing system, the momentary ratio of the operation of the aerobic/anaerobic energy-producing systems and/or the momentary ratio of the sympathetic/parasympathetic power of autonomic nervous system of the subject.

4. The method according to claim 1, further comprising:
repeatedly measuring the ambient temperature, and
the status of the aerobic and anaerobic energy-producing systems and/or the status of autonomic nervous system of the subject is further determined based on at least one ambient temperature measured.

5. The method according to claim 1, wherein the sensitivity of the temperature measurement is better than 0.1° C.

6. The method according to claim 1, wherein the output signal is output in the form of an audio signal and/or a visual signal and/or a vibration (haptic) signal.

7. The method according to claim 1, further comprising the measurement of performance intensity data characteristic to the activity, such as force, acceleration, power and speed.

8. The method according to claim 1, further comprising the measurement of other functional data of the body of the subject, such as the pulse rate.

9. The method according to claim 1, further comprising the transfer of processed data to a computer for processing, assessment and/or further analysis, optionally by a telemetric means.

10. The method according to claim 1, wherein
a) the sporting activity is running and monitoring involves the measurement of skin temperature, speed of forward motion and optionally the pulse rate of the runner;
b) the sporting activity is paddling and monitoring involves the measurement of skin temperature, power output by the paddle and optionally the pulse rate of the runner;
c) the sporting activity is cycling and monitoring involves the measurement of skin temperature, power output and optionally the pulse rate of the runner;
d) the sporting activity is swimming and monitoring involves the measurement of skin temperature, speed of forward motion and optionally the pulse rate of the runner;
e) the sporting activity is team sport and monitoring involves the measurement of skin temperature, the sum of average acceleration in unit time in the X, Y and Z directions and optionally the pulse rate of the runner.

11. The method according to claim 1, wherein the subject is human.

12. The method according to claim 1, wherein the subject is an animal.

13. The method according to claim 12, wherein the animal is a racing horse.

14. Device for indicating the status of the aerobic and anaerobic energy-producing systems and/or the status of autonomic nervous system of a human or animal subject during sporting, comprising:
means for repeatedly measuring the skin surface temperature of the subject; and
means for determining a temperature gradient of the skin temperature along time, based on the temperature data measured;
means for determining the momentary intensity of the aerobic energy-producing system, the momentary ratio of the operation of the aerobic/anaerobic energy-producing systems and/or the momentary ratio of the sympathetic/parasympathetic power of autonomic nervous system, based on the temperature gradient determined;
means for determining the status of the aerobic and anaerobic energy-producing systems and/or the status of autonomic nervous system of the subject, based on the momentary intensity of the aerobic energy-producing system, the momentary ratio of the operation of the aerobic/anaerobic energy-producing systems and/or the momentary ratio of the sympathetic/parasympathetic power of autonomic nervous system;
means for generating a signal indicative of the status of the aerobic and anaerobic energy-producing systems and/or the status of autonomic nervous system of the subject; and
means for outputting the signal relating to the subject in a sensible form.
